(19) United States
(12) Patent Application Publication
(10) Pub. No.: US 2025/0090748 A1
(43) Pub. Date: Mar. 20, 2025

(54) FLUID DELIVERY SYSTEMS AND METHODS FOR CONTINUOUS FLUID FLOW

(71) Applicant: IDEX Health & Science, LLC, Rohnert Park, CA (US)

(72) Inventor: John Barnacle, Petaluma, CA (US)

(73) Assignee: IDEX Health & Science, LLC, Rohnert Park, CA (US)

(21) Appl. No.: 18/825,476

(22) Filed: Sep. 5, 2024

Related U.S. Application Data
(60) Provisional application No. 63/538,576, filed on Sep. 15, 2023.

(51) Int. Cl. A61M 5/168

(52) U.S. Cl. CPC A61M 5/168

(57) ABSTRACT

A fluid delivery system for continuous fluid flow includes a first inlet in fluidic communication with a first pump, a first pressure sensor for detecting a first fluid pressure downstream from the first pump, a second inlet in fluidic communication with a second pump, a second pressure sensor for detecting a second fluid pressure downstream from the second pump, an outlet, a valve including a first position and a second position, wherein the first position fluidically connects the first inlet with the outlet, and the second position fluidically connects the first inlet, the second inlet, and the outlet, and a controller system including a processor and memory. The controller system is communicatively coupled with the first pump, the second pump, the first pressure sensor, the second pressure sensor, and the valve, wherein the controller system receives signals indicative of the first fluid pressure from the first pressure sensor and the second fluid pressure from the second pressure sensor, wherein the processor is programmed to direct the valve to move from the first position to the second position in response to the first fluid pressure being about equal to the second fluid pressure, and to substantially simultaneously cease fluid flow through the first inlet.

### CROSS-REFERENCE TO RELATED APPLICATION

This application claims the benefit of priority to U.S. Provisional Patent Application Ser. No. 63/538,576, filed Sep. 15, 2023, and entitled “Fluid Delivery Systems and Methods for Continuous Fluid Flow”, the contents of which being incorporated herein in their entirety.

### BACKGROUND

Valves, flow combiners, and flow splitters are typically utilized to control the passage of fluid(s) and may be used to control the flow rate of these fluid(s) along one or more fluid pathways. Depending on the type of valve utilized, the position of one or more valve components may be adjusted to achieve the desired delivery of fluid passing along the fluid pathway. In many applications, two or more input fluid streams are combined in some way to produce one output fluid stream. It is often desirable to provide fluid flow to downstream processes through this single output stream. Applications that use two or more pumps to drive the input flow stream often suffer from interrupted flow and/or pressure fluctuations to the combined output flow stream.

Typically, when two or more input fluid streams from alternating pumping mechanisms are attempted to be merged into one continuous output stream, a pressure pulsation or fluctuation occurs. These pressure pulsations are undesirable as they may decrease efficiency and accuracy of downstream processing and analysis. For example, analytical techniques such as high pressure liquid chromatography, mass spectrometry, and analytical separations devices are more accurate and efficient when provided with a continuous (uninterrupted) flow of fluid, without pressure pulsations or fluctuations. Accordingly, it is desirable to provide continuous fluid delivery to these downstream systems at a substantially constant pressure.

### SUMMARY

According to one aspect, a fluid delivery system for continuous fluid flow includes a first inlet in fluidic communication with a first pump, a first pressure sensor for detecting a first fluid pressure downstream from the first pump, a second inlet in fluidic communication with a second pump, a second pressure sensor for detecting a second fluid pressure downstream from the second pump, an outlet, a valve including a first position and a second position, wherein the first position fluidically connects the first inlet with the outlet, and the second position fluidically connects the first inlet, the second inlet, and the outlet, and a controller system including a processor and memory.

The controller system of the preceding paragraph is communicatively coupled with the first pump, the second pump, the first pressure sensor, the second pressure sensor, and the valve, wherein the controller system receives signals indicative of the first fluid pressure from the first pressure sensor and the second fluid pressure from the second pressure sensor, wherein the processor is programmed to direct the valve to move from the first position to the second position in response to the first fluid pressure being about equal to the second fluid pressure, and to substantially simultaneously cease fluid flow through the first inlet.

According to another aspect, a method of delivering fluid in a continuous fluid delivery system includes receiving a first fluid through a first inlet in fluidic communication with a first pump, receiving one or more signals from a first pressure sensor indicative of a first fluid pressure of the first fluid, receiving one or more signals from a second pressure sensor indicative of a second fluid pressure downstream from a second pump, comparing the first fluid pressure to the second fluid pressure, upon a determination that the first pressure is about equal to the second pressure, (i) operably controlling a valve sufficient to switch the valve from a first position to a second position, wherein the first position permits fluid flow of the first fluid through a system outlet and the second position fluidically connects the first pump, the second pump, and the system outlet, and (ii) ceasing fluid flow through the first inlet substantially simultaneously with switching the valve from the first position to the second position.

According to another aspect, a fluid delivery system for continuous fluid flow includes a first pump, a first pump pressure sensor for detecting fluid pressure associated with a chamber of the first pump, a second pump, a second pump pressure sensor for detecting fluid pressure associated with a chamber of the second pump, one or more mobile phase reservoirs, a shear valve including a rotor and a stator, the rotor being rotatable with respect to the stator about an axis, the rotor including a first rotor slot and the stator including a first stator orifice, a second stator orifice, a third stator orifice, a fourth stator orifice, wherein the first stator orifice is in fluid communication with the first pump, the second stator orifice is in fluid communication with the second pump, the third stator orifice is in fluid communication with the one or more mobile phase reservoirs, and the fourth stator orifice is in fluid communication with an outlet, and a controller system that includes a processor and memory, wherein the controller system is communicatively coupled with the first pump, the first pump pressure sensor, the second pump, the second pump pressure sensor, and the shear valve.

The fluid delivery system of the preceding paragraph may include the controller system receiving signals indicative of a first pressure from the first pump pressure sensor and a second pressure from the second pump pressure sensor, wherein the processor is capable of operably controlling the shear valve in response to the first pressure and the second pressure being about equal sufficient for the shear valve to rotate the rotor from a first position to a second position fluidically connecting the first rotor slot, the first stator orifice, the second stator orifice, and the outlet while operably controlling the first pump sufficient to substantially simultaneously cease fluid flow from the first pump.

According to another aspect, a method of controlling a shear valve for fluid delivery includes receiving one or more signals from a first pump pressure sensor indicative of a first pressure generated by a first pump, receiving one or more signals from a second pump pressure sensor indicative of a second pressure generated by a second pump, comparing the first pressure to the second pressure, upon a determination that the first pressure is about equal to the second pressure, operably controlling the shear valve sufficient to rotate a rotor from a first position to a second position fluidically connecting a first rotor slot, a first stator orifice, a second stator orifice, and an outlet, and substantially simultaneously with rotating the rotor to the second position, operably controlling the first pump to cease.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 illustrates a fluid delivery system 100 for continuous fluid flow, according to some embodiments.

FIG. 2 illustrates a method 200 of delivering continuous fluid flow, according to some embodiments.

FIG. 3 illustrates a fluid delivery system 300 for continuous fluid flow, according to some embodiments.

FIG. 4 illustrates a fluid delivery system 300 for continuous fluid flow, according to some embodiments.

FIG. 5 illustrates a fluid delivery system 300 for continuous fluid flow, according to some embodiments.

FIG. 6 illustrates a fluid delivery system 300 for continuous fluid flow, according to some embodiments.

FIG. 7 illustrates a fluid delivery system 300 for continuous fluid flow, according to some embodiments.

FIG. 8 illustrates a fluid delivery system 300 for continuous fluid flow, according to some embodiments.

FIG. 9 illustrates a fluid delivery system 300 for continuous fluid flow, according to some embodiments.

FIG. 10 illustrates a method of controlling a shear valve for continuous fluid delivery, according to some embodiments.

### DETAILED DESCRIPTION

Embodiments of the present disclosure provide novel systems and methods for providing continuous fluid flow to downstream systems. Many downstream systems require a single inlet for fluid(s). Typically, switching between two or more fluid sources at a flow combiner causes discontinuous flow due to pressure pulsations, undesirable variations in flow, and pressure differentials. Many systems, such as analytical devices, operate more accurately and efficiently when provided with a continuous flow of fluid. By controlling a valve or flow combiner based on certain process conditions, the systems and methods of the present disclosure can provide continuous fluid flow through a system outlet while switching between various fluid sources, such as two or more pumps.

As used herein, “continuous fluid flow” may refer to a substantially uninterrupted flow of one or more fluids. For example, continuous fluid flow may refer to a flow of fluid without pressure pulsations, flow noise, variations in flow, and/or pressure fluctuations.

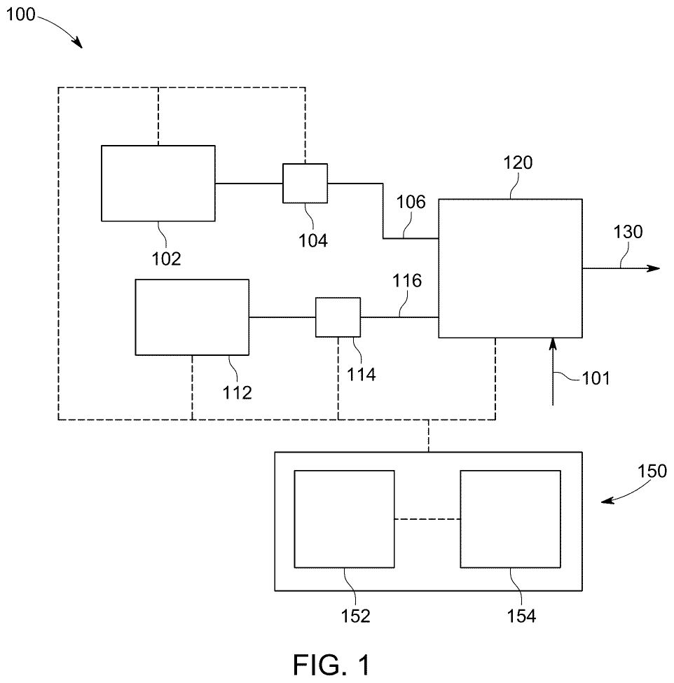

FIG. 1 illustrates a fluid delivery system 100 for continuous fluid flow, according to some embodiments. System 100 includes a system inlet 101, a first pump 102, a first pressure sensor 104, a first conduit 106, a second pump 112, a second pressure sensor 114, a second conduit 116, a valve 120, an output 130, and a controller system 150. Controller system 150 includes a processor 152 and memory 154. System 100 may be utilized for providing a continuous fluid flow to downstream processes. In one example, continuous fluid flow includes flow without pressure pulsations. A pressure pulsation may include a change in pressure of more than 0.1%, more than 0.5%, more than 1%, more than 3%, more than 5%, or values therebetween. Alternatively, a pressure pulsation may be measured in absolute pressure values. In one example, pressure pulsation refers to an increase/decrease in fluid pressure of greater than 0.01 bar. In another example, pressure pulsation refers to an increase/decrease in fluid pressure of greater than 0.1 bar. In yet another example, pressure pulsation refers to an increase/decrease in fluid pressure of greater than 1 bar.

In one example, system 100 is sufficient to provide continuous fluid flow while switching between two or more pumps and only using a single pump at a time. The operating pressure and flow rate of fluid in system 100 may be dependent on the downstream system. System 100 can provide continuous fluid flow through the outlet 130 without a programmed flow transition of fluid from first pump 102 and fluid from second pump 112. A programmed flow transition may include a transition in flow contribution from one pump to the other pump. A programmed flow transition may include providing flow from a first fluid source through an outlet while simultaneously providing flow from a second fluid source through the same outlet. Since no programmed flow transition is required, one pump may be dispensing while the other pump is aspirating.

First pump 102 may be in fluidic communication with one or more of first pressure sensor 104 and first conduit 106. First pump 102 may be filled and fluidically connected to an optional reservoir. In one example, fluidic communication includes connecting two or more components with a conduit, pipe, orifice, valve, and/or pump sufficient for fluid to flow between the two or more components. In another example, fluidic communication includes two or more components (that may or may not be physically connected), wherein fluid is able to flow between the two or more components. First pump 102 is capable of pressurizing a fluid, such as a first fluid (not shown), sufficient for delivering the fluid through outlet 130. In one example, first pump 102 includes a pump sufficient for solvent delivery. In another example, first pump 102 is a positive displacement pump, such as a reciprocating pump, a syringe pump, and a piston pump. First pump 102 may include a syringe pump including a pump chamber and a piston/plunger. The syringe pump may further include guide rods, a motor, and a lead screw.

First pressure sensor 104 may be downstream from first pump 102 and may be in fluidic communication with one or more of first pump 102 and first conduit 106. First pressure sensor 104 is capable of sending signals indicative of a pressure of the first fluid. Accordingly, first pressure sensor 104 is capable of sending signals indicative of first pump 102 outlet pressure. First pressure sensor 104 may measure the pressure in absolute, differential, and gauge pressure. In one example, first pressure sensor 104 is sufficient to measure pressure of one or more liquids. In another example, first pressure sensor 104 may include all sensors, transducers, and elements that are capable of producing an electrical signal proportional to a pressure or change in pressure. In yet another example, first pressure sensor 104 includes one or more of a strain gauge pressure sensor, a piezoelectric pressure sensor, and a capacitive pressure sensor.

Second pump 112 may be in fluidic communication with one or more of second pressure sensor 114 and second conduit 116. Second pump 112 may be filled and fluidically connected to an optional reservoir. This optional reservoir may be the same reservoir used for first pump 102. Second pump 112 is capable of pressurizing a fluid, such as a second fluid (not shown), sufficient for delivering the fluid through outlet 130. The first fluid and the second fluid may be the same fluid (same constitution) or different fluids. Accordingly, the first fluid and the second fluid may be of the same liquid constitution. In one example, second pump 112 includes a pump sufficient for solvent delivery. In another example, second pump 112 is a positive displacement pump, such as a reciprocating pump, a syringe pump, and a piston pump. Second pump 112 may include a syringe pump including a pump chamber and a piston/plunger. The syringe pump may further include guide rods, a motor, and a lead screw.

Second pressure sensor 114 may be downstream from second pump 112 and may be in fluidic communication with one or more of second pump 112 and second conduit 116. Second pressure sensor 114 is capable of sending signals indicative of a pressure of the second fluid. Accordingly, second pressure sensor 114 is capable of sending signals indicative of second pump 112 outlet pressure. Second pressure sensor 114 may measure the pressure in absolute, differential, and gauge pressure. In one example, second pressure sensor 114 is sufficient to measure pressure of one or more liquids. In another example, second pressure sensor 114 may include all sensors, transducers, and elements that are capable of producing an electrical signal proportional to a pressure or change in pressure. In yet another example, second pressure sensor 114 includes one or more of a strain gauge pressure sensor, a piezoelectric pressure sensor, and a capacitive pressure sensor.

Valve 120 may be in fluidic communication with one or more of system inlet 101, first pump 102, first pressure sensor 104, first conduit 106, second pump 112, second pressure sensor 114, second conduit 116, and the outlet 130. In one example, first conduit 106, second conduit 116, and outlet 130 are components of valve 120. Valve 120 may include a first position and a second position. The first position may fluidically connect first conduit 106 with outlet 130. The second position may fluidically connect first conduit 106, second conduit 116, and outlet 130. Valve 120 may further include a third position fluidically connecting second conduit 116 with outlet 130.

Valve 120 may include a valve driver for moving, rotating, or switching one or more components within valve 120. For example, the valve driver may be utilized to switch the position of the valve from the first position to the second position or from the second position to the first position. One or more of first pump 102 and second pump 112 may be upstream of valve 120. One or more of first pressure sensor 104 and second pressure sensor 114 may be upstream of valve 120.

Outlet 130 may be in fluidic communication with valve 120. Outlet 130 may deliver fluids pumped by one or more of first pump 102 and second pump 112 to downstream processes. Outlet 130 may be a portion of valve 120 and/or may be a conduit or pipe for delivering liquid. Outlet 130 may include a flow restriction that establishes a back pressure as a threshold to outlet flow. In one example, a pressure of at least 5 bar, at least 6 bar, at least 8 bar, or at least 10 bar is applied to outlet 130. System 100 may be utilized to provide continuous fluid flow to downstream equipment and processes. In one example, outlet 130 delivers fluid(s) to downstream processes such as reaction chambers, separations equipment, downstream pumps, tanks, and/or valves, chromatography equipment, and/or spectrometry equipment. In another example, outlet 130 delivers fluid(s) to downstream processes such as high performance liquid chromatography devices and mass spectrometry equipment. In yet another example, outlet 130 delivers fluid(s) to a mass spectrometer nebulizer.

Controller system 150 includes a processor 152 and memory 154. Processor 152 may be communicatively coupled to memory 154. Memory 154 may include non-transitory memory. Controller system 150 may be communicatively coupled with one or more of first pump 102, first pressure sensor 104, second pump 112, second pressure sensor 114, and the valve 120. Accordingly, processor 152 may be capable of operably controlling one or more of first pump 102, second pump 112, and valve 120. Controller system 150 may receive signals indicative of first fluid pressure from first pressure sensor 104, and controller system 150 may receive signals indicative of second fluid pressure from second pressure sensor 114.

Processor 152 is capable of operably controlling (and/or directing) valve 120 in response to the first fluid pressure being about equal to the second fluid pressure sufficient to switch/move the valve 120 from the first position to the second position, or from the third position to the second position. In one example, processor 152 is capable (operably) of substantially simultaneously ceasing fluid flow through the first conduit 106 when valve 120 is moved from the first position to the second position. Ceasing fluid flow through the first conduit 106 may include operably controlling the first pump 102 to cease. Similarly, processor 152 is capable (operably) of substantially simultaneously ceasing fluid flow through the second conduit 116 when valve 120 is moved to the second position.

Additionally, processor 152 is capable of operably controlling valve 120 in response to a signal from a valve position sensor adapted to detect the valve position. Processor 152 is capable of determining whether the first fluid pressure is about equal to the second fluid pressure based on a stored pressure differential condition. For example, processor 152 may compare the difference (such as a percent difference or absolute difference) in pressure between the first fluid pressure and the second fluid pressure to the stored pressure differential condition to determine if the pressures are about equal. In one example, if the difference in pressure between the first fluid pressure and the second fluid pressure is a value included in the stored pressure differential condition, then processor 152 determines that the pressures are about equal.

In some embodiments, the stored pressure differential condition may be a percentage value of differential pressure between the first and second fluid pressures. In one example, the stored pressure differential condition includes a percentage value between 0.01% and 10%. In another example, the stored pressure differential condition includes a percentage value between about 0.1% and about 5%. In yet another example, the stored pressure differential condition includes a percentage value between about 0.5% and about 2%. The stored pressure differential condition may include a percentage value less than 5%, less than 4%, less than 3%, less than 2%, or less than 1%.

In other embodiments, the stored pressure differential condition may include a range of values. In one example, the stored pressure differential condition includes values between −0.2 bar and +0.2 bar. In another example, the stored pressure differential condition includes values between −0.1 bar and +0.1 bar. In yet another example, the stored pressure differential condition includes values between −0.05 bar and +0.05 bar. For example, the stored differential condition may include values of about 0.2 bar, less than about 0.15 bar, less than about 0.1 bar, less than about 0.05 bar, less than about 0.01 bar, and/or 0.

Processor 152 is capable of operably controlling first pump 102 and/or second pump 112 based on timing the pump control with sending valve movement signals from controller system 150. In one example, processor 152 is capable of operably controlling first pump 102 sufficient to simultaneously cease fluid flow from first pump 102 when valve 120 is switched from first position to the second position. For example, ceasing first pump 102 substantially simultaneously may occur within 0.0001 seconds to 1 second of sending a signal to valve 120 to switch to the second position. Ceasing first pump 102 substantially simultaneously may occur within 0.001 seconds to 0.01 seconds of sending a signal to valve 120 to switch to the second position. Ceasing first pump 102 substantially simultaneously may occur within 0.01 seconds to 0.1 seconds of sending a signal to valve 120 to switch to the second position. Processor 152 is capable of similarly operably controlling second pump 112 based on these timing parameters.

Processor 152 is capable of operably controlling first pump 102 and/or second pump 112 based on the position of valve 120. For example, processor 152 is capable of operably controlling first pump 102 sufficient to simultaneously cease fluid flow from first pump 102 based on the precise location of one or more components in valve 120. Similarly, processor 152 is capable of operably controlling second pump 112 sufficient to simultaneously cease fluid flow from second pump 112 based on the precise location of one or more components in valve 120. The location of one or more components in valve 120 may be sensed by a position sensor located within valve 120.

Additionally, or alternatively, processor 152 is capable of operably controlling first pump 102 and/or second pump 112 based on signals from one or more of the first pressure sensor 104 and the second pressure sensor 114. During valve 120 position switching, a minor perturbation of pressure may be sensed by one or more of first pressure sensor 104 and second pressure sensor 114. By receiving and analyzing signals from one or more of the first pressure sensor 104 and second pressure sensor 114, processor 152 is capable of ceasing first pump 102 or second pump 112 based on a minor perturbation of pressure. Further, processor 152 is capable of operably controlling valve 120 sufficient to switch valve 120 from the first position to the second position to prevent over pressurizing one or more of first pump 102 (such as a chamber within first pump 102) and second pump 112 (such as a chamber within second pump 112).

Importantly, system 100 may be utilized to provide continuous fluid flow to downstream equipment and processes. Many downstream processes are more efficient and accurate when provided with a continuous fluid flow. By controlling a position of valve 120 based on process conditions, such as those indicated by first pressure sensor 104 and second pressure sensor 114, system 100 can provide a continuous fluid flow while switching between providing fluid flow from first pump 102 and second pump 112. By using two pumps, one pump can provide fluid flow while the other pump is not running or is aspirating.

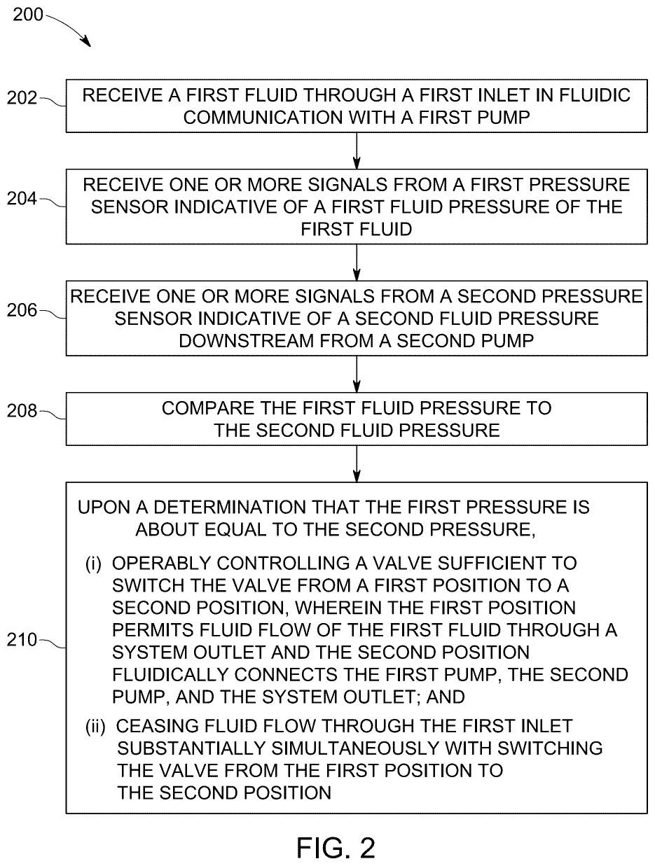

Referring to FIG. 2, a method 200 of delivering continuous fluid flow is illustrated according to some embodiments. Method 200 includes one or more of the following steps:

STEP 202, RECEIVE A FIRST FLUID THROUGH A FIRST INLET IN FLUIDIC COMMUNICATION WITH A FIRST PUMP, includes receiving a first fluid through a first inlet, such as first conduit 106, in fluidic communication with a first pump, such as first pump 102. In one example, the first fluid includes one or more liquid(s). In another example, the first fluid includes a solution of dissolved reagents in a fluid necessary for downstream processes. In yet another example, the first fluid includes a liquid solvent, such as one or more of methanol, acetone, ethanol, benzene, hexane, water, acetonitrile, and isopropanol. The first inlet may be a conduit between first pump 102 and valve 120.

STEP 204, RECEIVE ONE OR MORE SIGNALS FROM A FIRST PRESSURE SENSOR INDICATIVE OF A FIRST FLUID PRESSURE OF THE FIRST FLUID, includes receiving one or more signals from a first pressure sensor, such as first pressure sensor 104, indicative of the first fluid pressure of the first fluid. The signals may include electrical signals indicative of the first fluid pressure. In one example, the first pressure sensor is downstream from the first pump.

STEP 206, RECEIVE ONE OR MORE SIGNALS FROM A SECOND PRESSURE SENSOR INDICATIVE OF A SECOND FLUID PRESSURE DOWNSTREAM FROM A SECOND PUMP, includes receiving one or more signals from a second pressure sensor, such as second pressure sensor 114, indicative of a second fluid pressure downstream from a second pump, such as second pump 112. The signals may include electrical signals indicative of the second fluid pressure.

STEP 208, COMPARE THE FIRST FLUID PRESSURE TO THE SECOND FLUID PRESSURE, includes comparing, such as determining a difference value/percentage, between the first fluid pressure and the second fluid pressure. For example, a difference value of zero means that the first fluid pressure equals, or is about equal to, the second fluid pressure. To calculate the difference value, the second fluid pressure may be subtracted from the first fluid pressure, or vice versa. Comparing the first fluid pressure to the second fluid pressure may include determining that the first pressure is about equal to the second pressure. In one example, a controller system may determine that the first pressure and the second pressure are about equal if the difference percentage is between 0.01% and 10%. In another example, a controller system may determine that the first pressure and the second pressure are about equal if the difference percentage is between 0.1% and 5%. In yet another example, a controller system may determine that the first pressure and the second pressure are about equal if the difference value is between −1 bar and +1 bar. A controller system may determine that the first pressure and the second pressure are about equal if the difference value is less than about 0.2 bar, less than about 0.15 bar, less than about 0.1 bar, less than about 0.05 bar, less than about 0.01 bar, and/or 0.

STEP 210, UPON A DETERMINATION THAT THE FIRST PRESSURE IS ABOUT EQUAL TO THE SECOND PRESSURE, (i) OPERABLY CONTROLLING A VALVE SUFFICIENT TO SWITCH THE VALVE FROM A FIRST POSITION TO A SECOND POSITION, WHEREIN THE FIRST POSITION PERMITS FLUID FLOW OF THE FIRST FLUID THROUGH A SYSTEM OUTLET AND THE SECOND POSITION FLUIDICALLY CONNECTS THE FIRST PUMP, THE SECOND PUMP, AND THE SYSTEM OUTLET; AND (ii) CEASING FLUID FLOW THROUGH THE FIRST INLET SUBSTANTIALLY SIMULTANEOUSLY WITH SWITCHING THE VALVE FROM THE FIRST POSITION TO THE SECOND POSITION, includes operably controlling a valve, such as valve 120, sufficient to switch the valve from a first position to a second position. In one example, the first position permits fluid flow of the first fluid through a system outlet (such as outlet 130) and the second position fluidically connects the first pump (such as first pump 102), the second pump (such as second pump 112), and the system outlet (such as outlet 130). Alternatively, upon a determination that the first pressure is about equal to the second pressure, the valve may be switched from a third position to the second position. The third position may fluidically connect the second pump and the system outlet. Accordingly, fluid flow from the second pump may be ceased substantially simultaneously when the valve is switched from the third position to the second position.

Ceasing fluid flow through the first inlet substantially simultaneously with switching the valve from the first position to the second position may include operably controlling the first pump to cease. Method 200 may further include operably controlling the valve in response to a signal from a valve position sensor adapted to detect the valve position. In one example, the first pump is upstream of the valve. In another example, the second pump is upstream of the valve. In another example, method 200 may include a continuous flow of fluid without using two pumps simultaneously. Method 200 may include the use of system 100 and/or the steps of method 200 may be performed in any order.

Method 200 may provide fluid flow through the system outlet without a programmed flow transition of the first fluid and the second fluid. A programmed flow transition may include a transition in flow contribution from one pump to the other pump. A programmed flow transition may include providing flow from a first fluid source through an outlet while simultaneously providing flow from a second fluid source through the same outlet. For example, a programmed flow transition of the first fluid and the second fluid may include a 50/50 vol % mixture of the two fluids flowing through the outlet.

Importantly, system 100 and method 200 provide illustrative embodiments of systems and methods sufficient to provide continuous fluid flow through a single outlet, while switching the fluid source between two or more pumps. Further, in one example, only one pump needs to be providing fluid flow at any given time. This system and method can provide continuous fluid flow without pressure pulsations or fluctuations in the outlet. A continuous fluid flow is preferred since the outlet may be connected to downstream analytical devices. These analytical devices are not as efficient or accurate if fluid is provided with an interrupted flow and/or with pressure fluctuations.

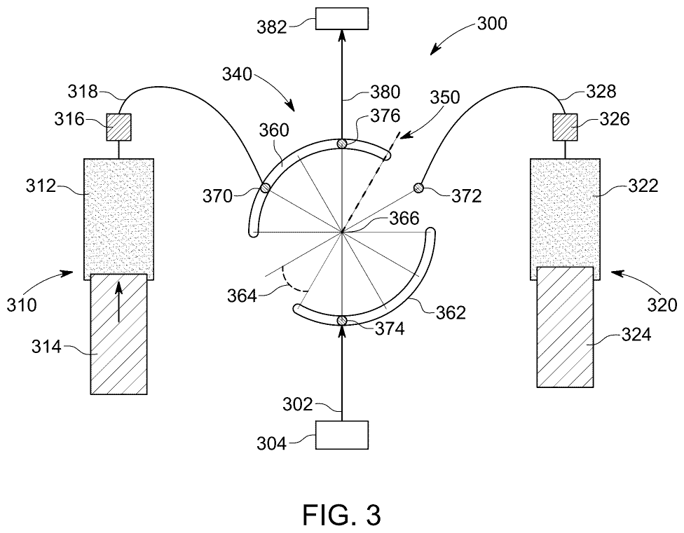

FIG. 3 illustrates a fluid delivery system 300 for continuous fluid flow, according to some embodiments. System 300 includes inlet 302, optional reservoir 304, first pump 310, first pressure sensor 316, first conduit 318, second pump 320, second pressure sensor 326, second conduit 328, shear valve 340, optional outlet conduit 380, and optional downstream system 382. In one example, first pump 310 includes first chamber 312 and first piston 314. In another example, second pump 320 includes second chamber 322 and second piston 324. First pump 310 and second pump 320 may be fluidically connected to first pressure sensor 316 and second pressure sensor 326, respectively. First pressure sensor 316 and second pressure sensor 326 may be on the outlet of, or downstream from, first pump 310 and second pump 320, respectively. First conduit 318 and second conduit 328 may fluidically connect first pump 310 and second pump 320 to shear valve 340, respectively. While shear valve 340 is in first position 350, first pump 310 may be dispensing fluid while second pump 320 is optionally pressurizing.

Shear valve 340 includes a rotor and a stator. The rotor may include and/or consist of two rotor slots. The rotor slots are sufficient to allow the passage of fluid through the rotor slot. These rotor slots may rotate about 12 positions based on 30-degree increments. The stator may include and/or consist of four stator orifices. The stator orifices are sufficient to allow the passage of fluid through the stator orifice. Accordingly, shear valve 340 includes one or more of a first rotor slot 360, a second rotor slot 362, a first stator orifice 370, a second stator orifice 372, a third stator orifice 374, and an fourth stator orifice 376. Shear valve 340 may include a valve driver (not shown) that is adapted to rotate the rotor. A controller may be configured to signal the valve driver to operate the rotor. The rotor may be rotatable with respect to the stator about an axis, such as axis 366. Therefore, one or more of first rotor slot 360 and second rotor slot 362 may be rotatable with respect to one or more of first stator orifice 370, second stator orifice 372, the third stator orifice 374, and fourth stator orifice 376. One or more of first rotor slot 360 and second rotor slot 362 may be rotated in 30-degree increments, as shown in reference numeral 364.

First stator orifice 370 is in fluid communication with first pump 310 and/or first pressure sensor 316. Second stator orifice 372 is in fluid communication with second pump 320 and/or second pressure sensor 326. Third stator orifice 374 is in fluid communication with inlet 302 and/or optional reservoir 304, such as one or more mobile phase reservoirs. The fourth stator orifice 376 may be in fluid communication with outlet conduit 380. Accordingly, fourth stator orifice 376 may be in fluid communication with optional downstream system 382. As shown in FIG. 3, shear valve 340 is shown in first position 350. In first position 350, first rotor slot 360 is fluidically connecting first stator orifice 370 and fourth stator orifice 376. In first position 350, second rotor slot 362 is in fluidic communication with third stator orifice 374 while second stator orifice 372 is isolated. In first position 350, first pump 310 may be providing fluid flow to outlet conduit 380. While first pump 310 provides fluid flow to outlet conduit 380, second pump 320 may be pressurizing fluid in second chamber 322 by moving/pushing second piston 324 into second chamber 322.

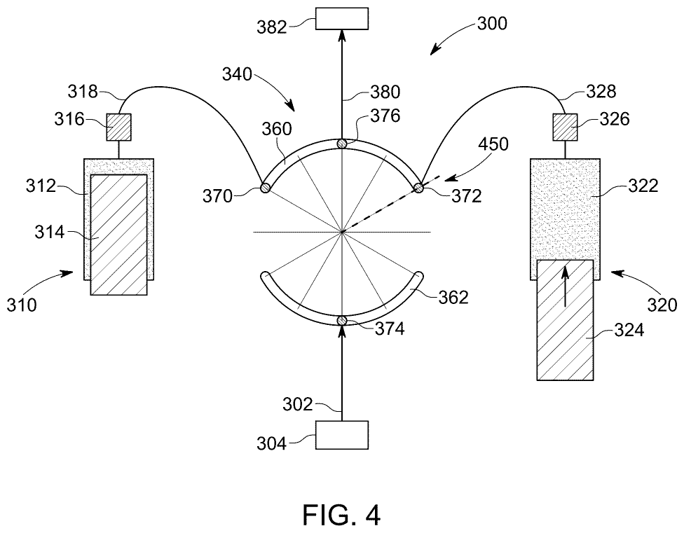

FIG. 4 illustrates a fluid delivery system 300 for continuous fluid flow, according to some embodiments. FIG. 4 illustrates shear valve 340 in second position 450. In second position 450, first rotor slot 360 fluidically connects first stator orifice 370, second stator orifice 372, and fourth stator orifice 376. In second position 450, second rotor slot 362 is in fluidic communication with third stator orifice 374. The valve driver may have rotated the rotor by 30-degrees (such as a clockwise rotation) from first position 350 to second position 450. In one example, in second position 450, first pump 310 or second pump 320 may be providing fluid flow to outlet conduit 380. For example, as shown in FIG. 4, second pump 320 is providing all fluid flow to outlet conduit 380 while shear valve 340 is in second position 450.

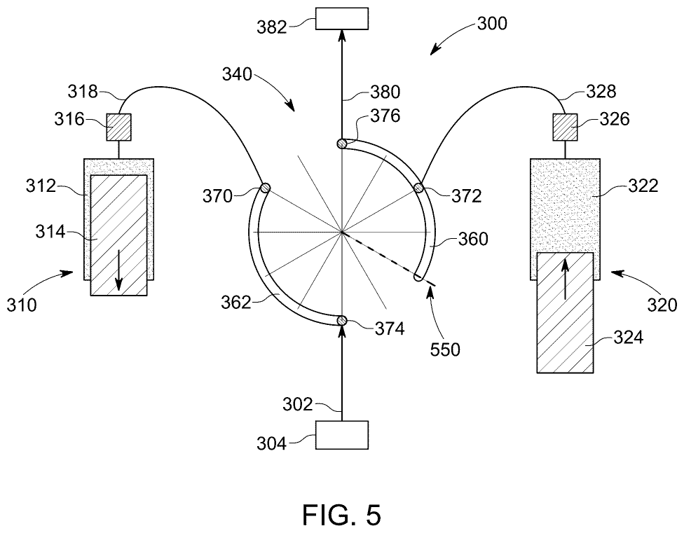

FIG. 5 illustrates a fluid delivery system 300 for continuous fluid flow, according to some embodiments. FIG. 5 illustrates shear valve 340 in third position 550. In third position 550, first rotor slot 360 is fluidically connecting second stator orifice 372 and fourth stator orifice 376. In third position 550, second rotor slot 362 is fluidically connecting first stator orifice 370 and third stator orifice 374. The valve driver may have rotated the rotor by 60-degrees (such as a clockwise rotation) from second position 450 to third position 550. In one example, in third position 550, second pump 320 is providing fluid flow to outlet conduit 380. In another example, in third position 550, first pump 310 may aspirating sufficient to fill first chamber 312 with fluid from optional reservoir 304. Importantly, the transition from second position 450 to third position 550 does not inhibit or alter the fluid flowing from second pump 320. The movement to third position 550 does allow first pump 310 to begin aspirating while second pump 320 is still providing fluid flow to fourth stator orifice 376. First pump 310 may aspirate at a higher rate compared to the dispense rate of second pump 320 in third position 550 to achieve full aspiration before second pump 320 is depleted of fluid.

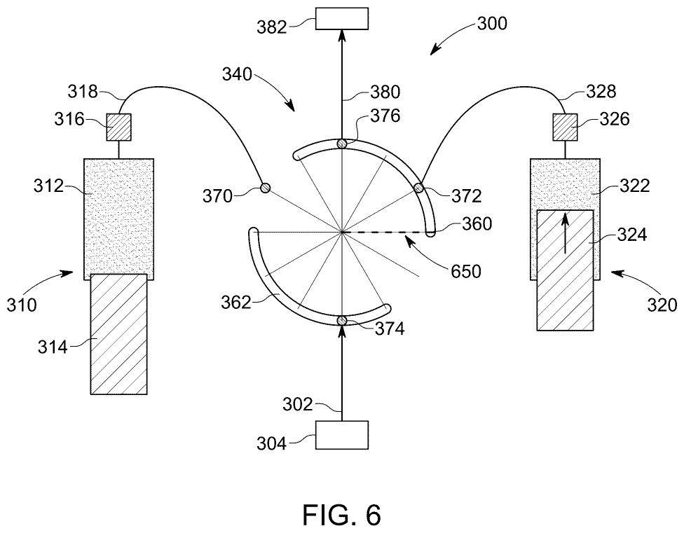

FIG. 6 illustrates a fluid delivery system 300 for continuous fluid flow, according to some embodiments. FIG. 6 illustrates shear valve 340 in fourth position 650. The valve driver may have rotated the rotor by 30-degrees (such as a counterclockwise rotation) from third position 550 to fourth position 650. In fourth position 650, first rotor slot 360 is fluidically connecting second stator orifice 372 and fourth stator orifice 376. In fourth position 650, second rotor slot 362 is in fluidic communication with third rotor slot 374. Additionally, in fourth position 650, first stator orifice 370 may be isolated. For example, first stator orifice 370 may be isolated from the rotor, such as from first rotor slot 360 and second rotor slot 362. Since first rotor orifice 370 may be isolated in fourth position 650, first pump 310 may be pressurizing in fourth position 650. In one example, first pump 310 pressurizes by moving first piston 314 into first chamber 312. While first pump 310 is pressurizing and/or after first pump 310 has pressurized, second pump 320 may be providing fluid flow to outlet conduit 380 through fourth stator orifice 376.

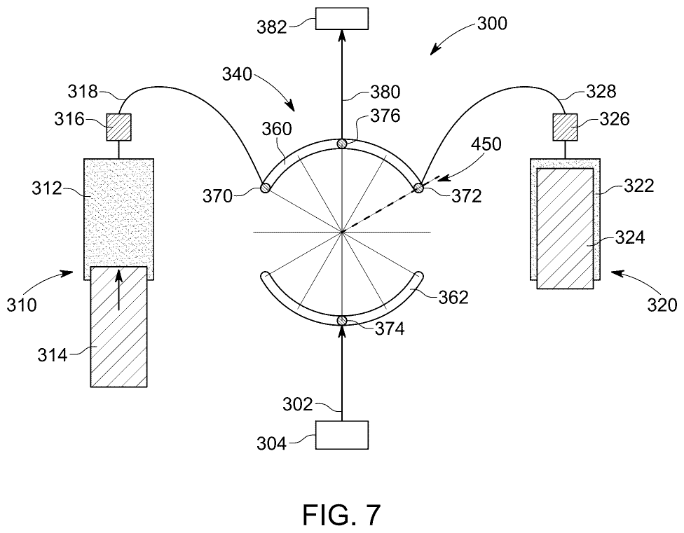

FIG. 7 illustrates a fluid delivery system 300 for continuous fluid flow, according to some embodiments. FIG. 7 illustrates shear valve 340 in second position 450. In second position 450, first rotor slot 360 fluidically connects first stator orifice 370, second stator orifice 372, and fourth stator orifice 376. In second position 450, second rotor slot 362 is in fluidic communication with third stator orifice 374. The valve driver may have rotated the rotor by 30-degrees (such as a clockwise rotation) from fourth position 650 to second position 450. In one example, in second position 450, first pump 310 or second pump 320 may be providing fluid flow to outlet conduit 380. For example, as shown in FIG. 7, first pump 310 is providing all fluid flow to outlet conduit 380 while shear valve 340 is in second position 450.

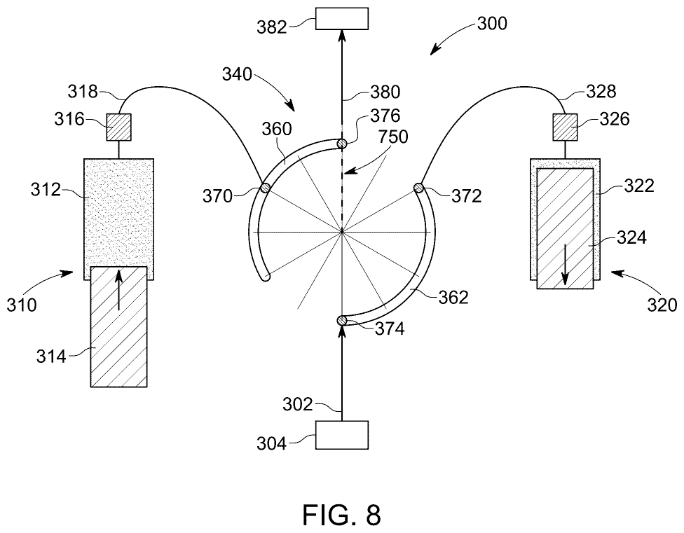

FIG. 8 illustrates a fluid delivery system 300 for continuous fluid flow, according to some embodiments. FIG. 8 illustrates shear valve 340 in fifth position 750. The valve driver may have rotated the rotor by 90-degrees (such as a counterclockwise rotation) from fourth position 650 to fifth position 750. In fifth position 750, first rotor slot 360 is fluidically connecting first stator orifice 370 and fourth stator orifice 376. In fifth position 750, second rotor slot 362 is fluidically connecting second stator orifice 372 and third stator orifice 374. In one example, in fifth position 750, first pump 310 is providing fluid flow to outlet conduit 380 through fourth stator orifice 376. In another example, in fifth position 750, second pump 320 may be aspirating sufficient to fill second chamber 322 with fluid from optional reservoir 304. The aspiration rate of second pump 320 may be higher than the dispense rate of first pump 310 so as to fully aspirate second pump 320 prior to first pump 310 fully dispensing all fluid from first chamber 312.

The valve driver is capable of rotating first rotor slot 360 and second rotor slot 362 to any of first position 350, second position 450, third position 550, fourth position 650, and fifth position 750. Accordingly, the valve driver is capable of rotating first rotor slot 360 and second rotor slot 362 to any of first position 350, second position 450, third position 550, fourth position 650, and fifth position 750 in any order. In one example, the valve driver is capable of rotating first rotor slot 360 and second rotor slot 362 from fifth position 750 to first position 350. In another example, the valve driver is capable of rotating first rotor slot 360 and second rotor slot 362 from fourth position 650 to second position 450. In one non-limiting example, the valve driver is capable of rotating first rotor slot 360 and second rotor slot 362 in the following order: first position 350, second position 450, third position 550, fourth position 650, second position 450, fifth position 750. The valve driver may be capable of rotating shear valve 340 from the fifth position 750 to first position 350 to restart the sequence.

System 300 is capable of operating at pressures consistent with downstream devices. In one example, system 300 is capable of operating at pressures between 1 bar and 500 bar. In another example, system 300 is capable of operating at pressures between 1 bar and 50 bar. In yet another example, system 300 is capable of operating at pressures between 1 bar and 10 bar. System 300 is capable of providing liquid flow to downstream devices at various flow rates. The downstream device may determine the desired liquid flow rate. In one example, system 300 is capable of providing liquid flow to downstream devices at a flow rate ranging from about 1 μL/min to 10 mL/min. System 300 is preferably configured to accommodate the flow rate needed for any particular application. The needs of the downstream system dictate the flow rate, and therefore the pumping capacity, valve pressure rating and slot size, and the like.

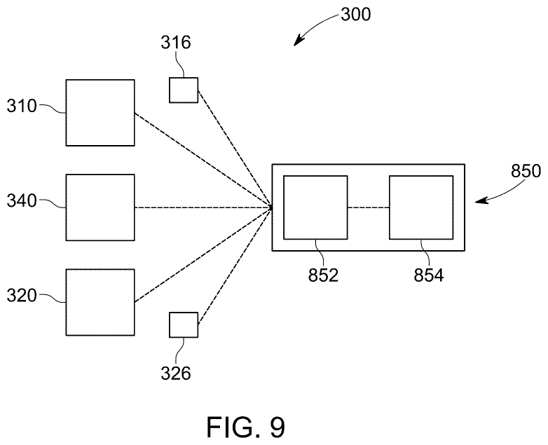

FIG. 9 illustrates a fluid delivery system 300 for continuous fluid flow, according to some embodiments. FIG. 9 illustrates that system 300 may include one or more controller systems 850 in communication with one or more other components in system 300. For example, controller system may include processor 852 and memory 854 (such as non-transitory memory) and may be communicatively coupled (such as with electrical communication) with one or more of first pump 310, first pressure sensor 316, second pump 320, second pressure sensor 326, and shear valve 340. Processor 852 may communicate with and access memory 854 sufficient to run a program or access system settings. Depending on the type of sensor, first pressure sensor 316 and second pressure sensor 326 may be in electrical communication with first pump 310 and second pump 320, respectively.

Controller system 850 is capable of receiving one or more signals from one or more components in system 300. For example, controller system 850 may receive signals indicative of a first pressure from first pressure sensor 316 and signals indicative of a second pressure from second pressure sensor 326. While a single controller system 850 is shown in FIG. 9, first pump 310, second pump 320, and shear valve 340 may each include individual controllers. These individual controllers may communicate with a parent controller such as controller system 850. In this example, controller system 850 will monitor pump and valve positions and outputs from pressure sensors. Controller system 850 may receive one or more signals from first pump 310 and second pump 320 indicative of pump position, pump speed, and/or pump pressure. For example, controller system 850 may receive one or more signals from first pump 310 and second pump 320 indicative of whether the individual pump is pumping, aspirating, pressurizing, and/or turned on or off. Additionally, controller system 850 is capable of receiving a signal from a valve position sensor adapted to detect rotor position of shear valve 340.

Processor 852 is capable of operably controlling shear valve 340 sufficient to rotate the rotor to third position 550 fluidically connecting first rotor slot 360, second stator orifice 372, and fourth stator orifice 376. Processor 852 is capable of operably controlling shear valve 340 sufficient to rotate the rotor to fourth position 650 fluidically connecting first rotor slot 360, second stator orifice 372, and fourth stator orifice 376 while isolating first stator orifice 370 from the rotor. Processor 852 is capable of operably controlling shear valve 340 sufficient to rotate the rotor to fifth position 750 fluidically connecting first rotor slot 360, first stator orifice 370, and fourth stator orifice 376. Processor 852 is capable of operably controlling shear valve 340 sufficient to rotate the rotor to fifth position 750 fluidically connecting second rotor slot 362, second stator orifice 372, and third stator orifice 374. Processor 852 is capable of operably controlling shear valve 340 in response to a signal from a valve position sensor adapted to detect rotor position. For example, the valve position sensor is adapted to detect if the rotor is in first position 350, second position 450, third position 550, fourth position 650, or fifth position 750.

Processor 852 is capable of operably controlling shear valve 340 in response to the first pressure being about equal to the second pressure sufficient to switch valve 340 from first position 350 to second position 450. Processor 852 is capable of determining whether the first pressure is about equal to the second pressure based on a stored pressure differential condition. For example, processor 852 may compare the difference in pressure between the first pressure and the second pressure to the stored pressure differential condition to determine if the pressures are about equal. In one example, if the difference in pressure between the first pressure and the second pressure is a value included in the stored pressure differential condition, then processor 852 determines that the pressures are about equal. Additionally, or alternatively, processor 852 is capable of operably controlling shear valve 340 based on the flow rates and volumes of first pump 310 and second pump 320. For example, processor 852 may operably control shear valve 340 to prevent a full pump depletion by receiving signals indicative of the flow rate of the first pump 310 and/or second pump 320.

In many embodiments, the stored pressure differential condition may be a percentage value. In one example, the stored pressure differential condition includes a percentage value between 0.01% and 10%. In another example, the stored pressure differential condition includes a percentage value between about 0.1% and about 5%. In yet another example, the stored pressure differential condition includes a percentage value between about 0.5% and about 2%. The stored pressure differential condition may include a percentage value less than 5%, less than 4%, less than 3%, less than 2%, or less than 1%.

In other embodiments, the stored pressure differential condition may include a range of values. In one example, the stored pressure differential condition includes values between −0.2 bar and +0.2 bar. In another example, the stored pressure differential condition includes values between −0.1 bar and +0.1 bar. In yet another example, the stored pressure differential condition includes values between −0.05 bar and +0.05 bar. For example, the stored pressure differential condition may include values of about 0.2 bar, less than about 0.15 bar, less than about 0.1 bar, less than about 0.05 bar, less than about 0.01 bar, and/or 0.

In one non-limiting example, if controller system 850 determines that the stored pressure differential condition is met, controller system 850 sends a command to a shear valve controller in communication with shear valve 340 to switch positions using the valve driver. Immediately following sending this command, controller system 850 may send a command to a pump controller in communication with first pump 310 or second pump 320 to stop first pump 310 or second pump 320. In another example, the next command controller system 850 sends is a signal to switch shear valve 340 to a position fluidically connecting first pump 310 or second pump 320 with third stator orifice 374.

Processor 852 is capable of operably controlling first pump 310 and/or second pump 320 based on timing the pump control with valve movement signals from controller system 850. In one example, processor 852 is capable of operably controlling first pump 310 sufficient to simultaneously cease fluid flow from first pump 310 when shear valve 340 is switched from first position 350 to the second position 450. For example, ceasing first pump 310 substantially simultaneously may occur within 0.0001 seconds to 1 second of sending a signal to shear valve 340 to switch to second position 450. Ceasing first pump 310 substantially simultaneously may occur within 0.001 seconds to 0.01 seconds of sending a signal to shear valve 340 to switch to second position 450. Ceasing first pump 310 substantially simultaneously may occur within 0.01 seconds to 0.1 seconds of sending a signal to shear valve 340 to switch to the second position 450. Processor 852 is capable of similarly operably controlling second pump 320 based on these timing parameters.

Processor 852 is capable of operably controlling first pump 310 and/or second pump 320 based on the position of shear valve 340. Processor 852 is capable of operably controlling first pump 310 sufficient to simultaneously cease fluid flow from first pump 310 based on the precise location of one or more components in shear valve 310. For example, processor 852 can control first pump 310 sufficient to simultaneously cease fluid flow from first pump 310 based on the position of first rotor slot 360 and/or the position of second rotor slot 362 in relation to one or more of first stator orifice 370, second stator orifice 372, third stator orifice 374, and fourth stator orifice 376. The location of one or more components in the valve may be sensed by a position sensor located within the valve. In one example, the position sensor is capable of sensing when a rotor is starting to move across an orifice.

Additionally, or alternatively, processor 852 is capable of operably controlling first pump 310 and/or second pump 320 based on signals from one or more of first pressure sensor 316 and second pressure sensor 326. During shear valve 340 position switching, a minor perturbation of pressure may be sensed by one or more of first pressure sensor 316 and second pressure sensor 326. By receiving and analyzing signals from one or more of the first pressure sensor 316 and second pressure sensor 326, processor 852 is capable of ceasing first pump 310 or second pump 320 based on a minor perturbation of pressure. Similarly, processor 852 is capable of operably controlling second pump 320 based on sensed pressure perturbations. Further, processor 852 is capable of operably controlling shear valve 340 sufficient to switch shear valve 340 from first position 350 to second position 450 to prevent over pressurizing one or more of first pump 310 (such as first chamber 312) and second pump 320 (such as second chamber 322).

Similarly, processor 852 is capable of operably controlling second pump 320 when the stored pressure differential condition is met at fourth position 650. Processor 852 may send a signal to the valve driver to switch shear valve 340 from fourth position 650 to second position 450. As shear valve 340 is switched from fourth position 650 to second position 450, second pump 320 may be controlled to stop dispensing by a command from controller system 850. In one example, processor 852 is capable of operably controlling second pump 320 sufficient to simultaneously cease fluid flow from second pump 320 based on the precise location of one or more components in shear valve 340.

Importantly, system 300 is capable of efficiently providing continuous flow to downstream processes. Compared to check valves that may suffer from undesirable variations in flow when switching between pumps, system 300 is capable of switching between providing flow from first pump 310 and second pump 320 without pressure pulsations. Unlike a programmed flow transition where two pumps are both providing flow to downstream systems, system 300 can provide continuous flow while only providing flow from one pump at a time. These flow transitions often require numerous, expensive controllers and precise pump speed and/or flow rate monitoring to control the transition. By providing a continuous flow stream using the controller system and shear valve of the present disclosure, downstream processes such as analytical equipment can operate more accurately and efficiently.

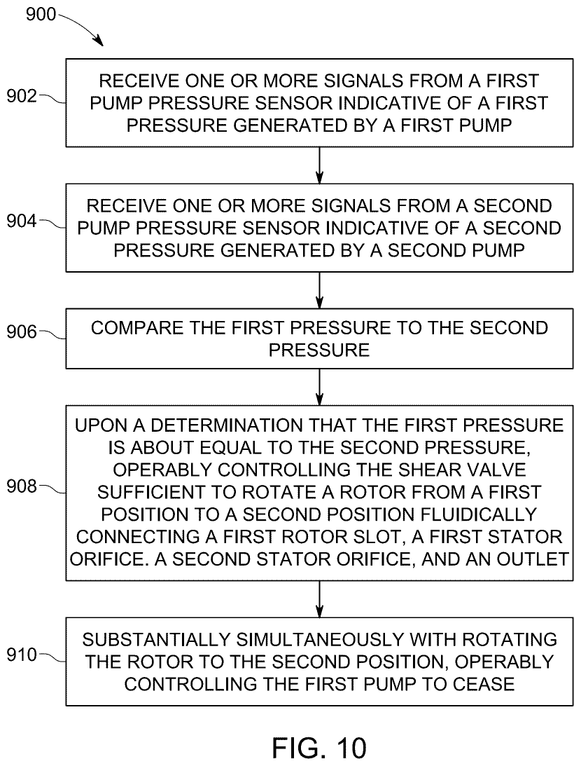

Referring to FIG. 10, a method 900 of controlling a shear valve for continuous fluid delivery is illustrated according to some embodiments. Method 900 includes one or more of the following steps:

STEP 902, RECEIVE ONE OR MORE SIGNALS FROM A FIRST PUMP PRESSURE SENSOR INDICATIVE OF A FIRST PRESSURE GENERATED BY A FIRST PUMP, includes receiving one or more signals from a first pump pressure sensor (such as first pressure sensor 316) indicative of a first pressure generated by a first pump (such as first pump 310). The signals may include electrical signals indicative of the first pressure.

STEP 904, RECEIVE ONE OR MORE SIGNALS FROM A SECOND PUMP PRESSURE SENSOR INDICATIVE OF A SECOND PRESSURE GENERATED BY A SECOND PUMP, includes receiving one or more signals from a second pump pressure sensor (such as second pressure sensor 326) indicative of a second pressure generated by a second pump (such as second pump 320). The signals may include electrical signals indicative of the second pressure.

STEP 906, COMPARE THE FIRST PRESSURE TO THE SECOND PRESSURE includes comparing, such as determining a difference value/percentage, between the first pressure and the second pressure. For example, a difference value of zero means that the first pressure equals, or is about equal to, the second pressure. To calculate the difference value, the second pressure may be subtracted from the first pressure, or vice versa. Comparing the first pressure to the second pressure may include determining that the first pressure is about equal to the second pressure.

The controller system may determine that the first pressure and the second pressure are about equal if the difference percentage equals a stored pressure differential condition. In many embodiments, the stored pressure differential condition may be a percentage value. In one example, the stored pressure differential condition includes a percentage value between 0.01% and 10%. In another example, the stored pressure differential condition includes a percentage value between about 0.1% and about 5%. In yet another example, the stored pressure differential condition includes a percentage value between about 0.5% and about 2%. The stored pressure differential condition may include a percentage value less than 5%, less than 4%, less than 3%, less than 2%, or less than 1%.

In other embodiments, a controller system may determine that the first pressure and the second pressure are about equal if the difference value equals a stored pressure differential condition between −0.2 bar and +0.2 bar. In another example, a controller system may determine that the first pressure and the second pressure are about equal if the difference value equals a stored pressure differential condition between −0.1 bar and +0.1 bar. In yet another example, a controller system may determine that the first pressure and the second pressure are about equal if the difference value equals a stored pressure differential condition between −0.05 bar and +0.05 bar. A controller system may determine that the first pressure and the second pressure are about equal if the difference value is less than about 0.2 bar, less than about 0.15 bar, less than about 0.1 bar, less than about 0.05 bar, less than about 0.01 bar, and/or 0.

STEP 908, UPON A DETERMINATION THAT THE FIRST PRESSURE IS ABOUT EQUAL TO THE SECOND PRESSURE, OPERABLY CONTROLLING THE SHEAR VALVE SUFFICIENT TO ROTATE A ROTOR FROM A FIRST POSITION TO A SECOND POSITION FLUIDICALLY CONNECTING A FIRST ROTOR SLOT, A FIRST STATOR ORIFICE, A SECOND STATOR ORIFICE, AND AN OUTLET, includes operably controlling the shear valve, such as shear valve 340, sufficient to rotate a rotor from a first position to a second position, such as second position 450, fluidically connecting a first rotor slot, a first stator orifice, a second stator orifice, and an outlet. The first rotor slot may be first rotor slot 360. The first stator orifice may be first stator orifice 370, the second stator orifice may be second stator orifice 372, and the outlet may be fourth stator orifice 376 or outlet conduit 380.

STEP 910, SUBSTANTIALLY SIMULTANEOUSLY WITH ROTATING THE ROTOR TO THE SECOND POSITION, OPERABLY CONTROLLING THE FIRST PUMP TO CEASE, including substantially simultaneously with rotating the rotor to the second position, such as second position 450, operably controlling the first pump, such as first pump 310, to cease.

Ceasing may include sending a signal to the pump to stop movement or rotation of the pump. In one example, ceasing includes turning the pump off. In another example, ceasing includes stopping output flow of fluid from a pump. For example, ceasing the first pump substantially simultaneously may occur within 0.0001 seconds to 1 second of switching to the second position. Ceasing the first pump substantially simultaneously may occur within 0.001 seconds to 0.01 seconds of switching to the second position. Ceasing the first pump substantially simultaneously may occur within 0.01 seconds to 0.1 seconds of switching to the second position. Ceasing the first pump substantially simultaneously may occur at exactly the same time as switching to the second position. Alternatively, the second pump may be operably controlled to cease substantially simultaneously with rotating the rotor to the second position. The steps of method 900 may be performed in any order.

Operably controlling the first pump to cease may include controlling the first pump based on rotor position. The location of one or more components in the valve may be sensed by a position sensor located within the valve. In one example, the position sensor is capable of sensing when a rotor is starting to move across an orifice. In one example, the first pump is controlled to cease once the valve position sensor has sensed that the valve is in the second position. Additionally, or alternatively, the first pump is controlled to cease if a minor pressure perturbation is sensed by one or more of the first pressure sensor and the second pressure sensor.

Importantly, method 900 provides continuous fluid flow to one or more downstream devices while switching the fluid stream. Accordingly, one pump may provide 100% of the fluid flow to the downstream devices at all times. This allows the secondary pump to aspirate from a reservoir while the primary pump is dispensing. Further, method 900 provides continuous fluid flow without pressure pulsations and/or programmed flow transitions. Downstream devices operate more accurately and efficiently when provided with a continuous fluid flow.

While the disclosure has been described with reference to an exemplary embodiment(s), it will be understood by those skilled in the art that various changes may be made, and equivalents may be substituted for elements thereof without departing from the scope of the embodiment(s). In addition, many modifications may be made to adapt a particular situation or material to the teachings of the embodiment(s) without departing from the essential scope thereof. Therefore, it is intended that the disclosure is not limited to the disclosed embodiment(s), but that the disclosure will include all embodiments falling within the scope of the appended claims. Various examples have been described. These and other examples are within the scope of the following claims.

What is claimed is:

1. A fluid delivery system for continuous fluid flow comprising:
a first inlet in fluidic communication with a first pump;
a first pressure sensor for detecting a first fluid pressure downstream from the first pump;
a second inlet in fluidic communication with a second pump;
a second pressure sensor for detecting a second fluid pressure downstream from the second pump;
an outlet;
a valve including a first position and a second position, wherein the first position fluidically connects the first inlet with the outlet, and the second position fluidically connects the first inlet, the second inlet, and the outlet; and
a controller system including a processor and memory, wherein the controller system is communicatively coupled with the first pump, the second pump, the first pressure sensor, the second pressure sensor, and the valve,
wherein the controller system receives signals indicative of the first fluid pressure from the first pressure sensor and the second fluid pressure from the second pressure sensor,
wherein the processor is programmed to direct the valve to move from the first position to the second position in response to the first fluid pressure being about equal to the second fluid pressure, and to substantially simultaneously cease fluid flow through the first inlet.

2. The system of claim 1, wherein the processor determines whether the first fluid pressure is about equal to the second fluid pressure by comparing a difference between the first and second fluid pressures to a stored pressure differential value.

3. The system of claim 1, wherein the processor is capable of operably controlling the valve in response to a signal from a valve position sensor adapted to detect the valve position.

4. The system of claim 1, wherein ceasing flow through the first inlet includes operably controlling the first pump to cease.

5. The system of claim 1, wherein the first pump and the second pump are upstream of the valve.

6. The system of claim 1, wherein the first fluid and the second fluid are the same constitution.

7. A method of delivering fluid in a continuous fluid delivery system, the method comprising:
receiving a first fluid through a first inlet in fluidic communication with a first pump;
receiving one or more signals from a first pressure sensor indicative of a first fluid pressure of the first fluid;
receiving one or more signals from a second pressure sensor indicative of a second fluid pressure downstream from a second pump;
comparing the first fluid pressure to the second fluid pressure; and
upon a determination that the first pressure is about equal to the second pressure,
(i) operably controlling a valve sufficient to switch the valve from a first position to a second position, wherein the first position permits fluid flow of the first fluid through a system outlet and the second position fluidically connects the first pump, the second pump, and the system outlet; and
(ii) ceasing fluid flow through the first inlet substantially simultaneously with switching the valve from the first position to the second position.

8. The method of claim 7 wherein ceasing fluid flow through the first inlet includes operably controlling the first pump to cease.

9. The method of claim 7 including operably controlling the valve in response to a signal from a valve position sensor adapted to detect the valve position.

10. The method of claim 7, wherein the first pump and the second pump are upstream of the valve.

11. The method of claim 7, including determining whether the first pressure is about equal to the second pressure by comparing a difference between the first and second pressures to a stored pressure differential value.

12. A method of controlling a shear valve for fluid delivery comprising:
receiving one or more signals from a first pump pressure sensor indicative of a first pressure generated by a first pump;
receiving one or more signals from a second pump pressure sensor indicative of a second pressure generated by a second pump;
comparing the first pressure to the second pressure;
upon a determination that the first pressure is about equal to the second pressure, operably controlling the shear valve sufficient to rotate a rotor from a first position to a second position fluidically connecting a first rotor slot, a first stator orifice, a second stator orifice, and an outlet; and
substantially simultaneously with rotating the rotor to the second position, operably controlling the first pump to cease.

13. The method of claim 12, wherein the processor is capable of operably controlling the shear valve in response to a signal from a valve position sensor adapted to detect the rotor position.

14. The method of claim 12, wherein operably controlling the first pump to cease causes fluid flow from the first pump to cease once the rotor has been rotated to the second position.

15. The method of claim 12, wherein the shear valve further includes a third stator orifice.

16. The method of claim 15, wherein the third stator orifice is in fluid communication with a second rotor slot in the second position.

17. The method of claim 15, wherein the processor is capable of operably controlling the shear valve sufficient to rotate the rotor to a third position fluidically connecting the second stator orifice and the outlet.

18. The method of claim 12, wherein the first pump and the second pump are selected from a reciprocating pump and a syringe pump.

19. The method of claim 12, wherein the first pump and the second pump are upstream of the shear valve.

20. The method of claim 12, including determining whether the first pressure is about equal to the second pressure based on a stored pressure differential condition.
